(19) United States
(12) Patent Application Publication
(10) Pub. No.: US 2007/0130642 A1
(43) Pub. Date: Jun. 7, 2007

(54) Methods and compositions for reducing the expression of a polynucleotide of interest

(75) Inventor: Kimberly F. Glassman, Ankeny, IA (US)

Correspondence Address: ALSTON & BIRD LLP;PIONEER HI-BRED INTERNATIONAL, INC., BANK OF AMERICA PLAZA, 101 SOUTH TRYON STREET, SUITE 4000, CHARLOTTE, NC, 28280-4000, US

(73) Assignee: Pioneer Hi-Bred International, Inc., Johnston, IA (US)

(21) Appl. No.: 11/598,899

(22) Filed: Nov. 14, 2006

Related U.S. Application Data
(60) Provisional application No. 60/736,453, filed on Nov. 14, 2005.

(51) Int. Cl. A01H 1/00; C12N 15/82; C12N 5/04

(52) U.S. Cl. 800/278; 435/468; 435/419

(57) ABSTRACT

The present invention provides methods and compositions that allow for the modulation of the level of at least two polynucleotides or the polypeptides encoded thereby in a plant or plant part. Specifically, the present invention relates to recombinant polynucleotides that are designed to allow for the overexpression of a polynucleotide of interest and also elicits a specific silencing effect on a second polynucleotide of interest. In specific embodiments, the level of at least two agronomically important sequences is modulated. Recombinant polynucleotides capable of eliciting these effects, as well as, plants, plant parts, seeds and grain having the recombinant polynucleotides are also provided.

### CROSS-REFERENCE TO RELATED APPLICATIONS

This application claims the benefit of U.S. Provisional Application No. 60/736,453, filed on Nov. 14, 2005, herein incorporated by reference in its entirety.

### FIELD OF THE INVENTION

The invention relates to the genetic modification of plants. In particular, methods and compositions are provided for reducing the expression level of a polynucleotide in a plant or plant part.

### BACKGROUND OF THE INVENTION

Methods for gene silencing that exploit the RNA silencing pathways in plants are playing an increasingly important role in analyzing and manipulating gene function in a sequence-specific manner. In fact, RNA silencing has been employed to modulate various aspects of plant development. Often, the art desires to modulate the level of more than one polynucleotide in a plant. In such instances, the art frequently employs individual trait gene cassettes each having a separate promoter. In cases where multiple genes need to be silenced in a given tissue, gene fusions have been effective in directing such silencing. Likewise, there has been the possibility of creating fusions of some coding sequences for overexpression of sequences of interest. Using individual expression cassettes results in a large vector size which increases the regulatory effort to sequence and characterized the vectors. In addition, in any given transgenic event, any given cassette is expressed at a level independent of the other cassette in the same vector. This frequently means that many events must be generated and screened to identify the few where all cassettes are expressed at the desired level.

Methods are needed in the art that allow for smaller vectors, with better coordination of expression levels for traits of interest, regardless of whether they are for overexpression or for silencing.

### BRIEF SUMMARY OF THE INVENTION

The present invention provides methods and compositions that allow for the modulation of the expression of at least two polynucleotides or the polypeptides encoded thereby in a plant or plant part. Specifically, the present invention relates to recombinant polynucleotides that are designed to allow for the overexpression of a polynucleotide of interest and also elicit a specific silencing effect on a second polynucleotide of interest.

Compositions of the invention comprise a recombinant polynucleotide comprising a first agronomically important polynucleotide comprising an intron comprising a silencing element. The intron has the following characteristics: it is capable of being spliced in a plant from the first agronomically important polynucleotide; upon splicing of the intron, the expression of the first agronomically important polynucleotide is increased; and, the intron is capable of reducing the expression of a second agronomically important polynucleotide or a polypeptide encoded thereby. In specific embodiments, the silencing element is selected from the group consisting of a sense suppression element, an antisense suppression element or a hairpin suppression element.

Methods are provided for modulating the expression of at least two polynucleotides of interest in a plant. The method comprises introducing into the plant a recombinant polynucleotide comprising a first polynucleotide of interest comprising an intron comprising a silencing element. The intron has the following characteristics: it is capable of being spliced from the first polynucleotide of interest in the plant; upon splicing of the intron, the expression of the first polynucleotide of interest is increased; and, the intron is capable of reducing the expression of a second polynucleotide of interest or a polypeptide encoded thereby. The recombinant polynucleotide is expressed in the plant or a plant part and thereby increases the expression of the first polynucleotide of interest and decreases the expression of the second polynucleotide of interest or a polypeptide encoded thereby. In specific embodiments, the second polynucleotide of interest confers an agronomically important trait. In other embodiments, the first and the second polynucleotide of interest confers an agronomically important trait.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

I. Compositions

Recombinant polynucleotides are provided that are designed to allow for the overexpression of a polynucleotide of interest and also elicit a specific silencing effect on a second polynucleotide of interest. The recombinant polynucleotide comprises a first polynucleotide of interest comprising an intron comprising a silencing element. Upon expression of the polynucleotide of interest, the cellular splicing machinery splices the intron from the polynucleotide. The spliced intron comprising the silencing element is capable of reducing or eliminating the level of a target polynucleotide, while the first polynucleotide is overexpressed. This design allows for the generation of smaller vectors with better coordination of expression levels for the traits of interest, regardless of whether they are for overexpression or for silencing.

A. Intron Sequences

The recombinant polynucleotide provided by the present invention comprises an intron, where the intron comprises a silencing element. When the recombinant polynucleotide is-expressed in a plant, the intron is spliced from the pre-mRNA transcript. The intron is then capable of decreasing the level of a polynucleotide of interest (or the polypeptide encoded thereby) that the silencing element is designed to target. As defined herein, an “intron” comprises a polynucleotide sequence which, when contained in the context of a pre-mRNA, is recognized by the splicing machinery and spliced from the pre-mRNA to form a spliced mRNA (i.e., the intron is splicing competent). See, for a review of pre-mRNA splicing in plants, Simpson et al. (1996) Plant Molecular Biology 32:1-41; Brown et al. (1998) Annu. Rev. Plant Physiol. Plant Mol. Biol. 49:77-95; Schuler et al. (1998) Plant Pre-mRNA Splicing, Bailey-Serres, J. and Gallie, D. R., eds, 1-19, American Society of Plant Physiologist; each of which is herein incorporated by reference, for a review of pre-mRNA splicing in plants. The structural requirements of a splicing competent intron are well characterized in the art. See, for example Zdravko et al. (2000) Trends in Plant Science 5:160-7, herein incorporated by reference. In general, an intron comprises various spliceosome-dependant nucleotide elements which comprise the splice donor site (5′ splice site), the acceptor site (3′ splice site), and the branch point domain. It is recognized that exon sequences may also influence the ability of the intron to be recognized by the splicing machinery and allow the intron to be removed from the transcript. The intron employed can be naturally occurring or synthetically derived.

A donor splice site and the acceptor splice site represent the boundaries of the intron sequence. The consensus sequence for a donor splice site in higher plants comprises AG/GUAAGU (SEQ ID NO:1), where the underlined nucleotides form part of the exon and the slash indicates the splice point. The sequence of the donor splice sites found in plants can deviate significantly from the consensus sequence set forth in SEQ ID NO:1. In fact, a functional variant of plant donor sites can vary from the consensus set forth in SEQ ID NO:1 by 1, 2, 3, 4, 5, 6, or 7 nucleotides. Representative functional donor splice sites that function in plants are known. See, Zdravko et al. (2000) Trends in Plant Science 5:160-7; McCullough et al. (1993) Mol. Cell. Biol. 13:1323-1331; Egoavil et al. (1997) Plant J 12:971-80; Goodall et al. (1990) Plant Mol. Biol. 14:727-733; Filipowicz et al. (1994) Pre-mRNA Processing, Lamand A, ed. RG Landes Publishers, Georgetown Publishers; and Brendel et al. (1998) Nucleic Acid Research 26:4748-4757.

The consensus sequence for an acceptor splice site in higher plants is UGCAG/G (SEQ ID NO:2), where the underlined nucleotide represent an exon sequence and the slash represent the splice point. The sequence of the acceptor splice sites found in plants can deviate significantly from the consensus sequence set forth in SEQ ID NO:2. In fact, functional variant plant acceptor sites can vary from the consensus set forth in SEQ ID NO:2 by 1, 2, 3, 4, 5, or 6 nucleotides. Representative functional acceptor splice sites that function in plants can be found for example, in Hebsgaard et al. (1996) Nucleic Acid Research 3492-3452; Lal et al. (1999) Plant Physiol. 120: 65-72; Brown et al. (1986) Nucleic Acid Research 24:9549-9559, each of which is herein incorporated by reference.

The branch point has a loose consensus sequence of CURAY (SEQ ID NO:3) and is found about 30 nucleotides upstream of the AG nucleotide. The sequence of the branch point domain found in plants can deviate significantly from the consensus sequence set forth in SEQ ID NO:3. In fact, functional variant plant acceptor sites can vary from the consensus set forth in SEQ ID NO:3 by 1, 2, 3, or 4 nucleotides. Representative functional branch point domains that function in plants can be found for example, in Brown et al. (1986) Nucleic Acid Research 24:9549-9559 and Tolstrup et al. (1997) Nucleic Acid Research 15:3159-3163 and Vogel et al. (1997) Nucleic Acid Research 25:2030-2031, each of which is herein incorporated by reference.

Methods are known in the art to determine if an intron is functional (i.e., splicing competent). For example, the intron can be interested into a DNA construct and expressed, in vivo or in vitro, under conditions where pre-mRNA splicing is known to occur. Spliced and/or unspliced product can be detected using standard techniques including, for example, Northern analysis, Western analysis, or assaying for the appropriate protein activity.

The intron employed in the methods and compositions of the invention can be of any length, so long as it is functional in a plant. For example, the intron can be about 40 to about 300 nucleotides in length or greater or about 25 to about 1000 nucleotides, about 60 to 150 nucleotides, about 75 to about 275 nucleotides, or about 50 to about 200 nucleotides. In specific embodiments, the intron is about 25 nt, about 30 nt, about 40 nt, about 50 nt, about 60 nt, about 70 nt, about 80 nt, about 90 nt, about 100 nt, 110 nt, about 120 nt, about 130 nt, about 140 nt, about 150 nt, about 160 nt, about 170 nt, about 180 nt, about 190 nt, about 200 nt, about 210 nt, about 220 nt, about 230 nt, about 240 nt, about 250 nt, about 260 nt, about 270 nt, about 280 nt, about 290 nt, about 300 nt, about 350 nt, about 400 nt, about 450 nt, about 500 nt, about 550 nt, about 600 nt, about 650 nt, about 700 nt or greater.

In addition, the intron employed in the methods and compositions of the invention can have a compositional bias and have a higher A+T content than the surrounding exon sequences. For example, the G+C content of the surrounding exons can exceeds the G+C content of the intron by at least 1%, 2%, 3%, 4%, 5%, 6%, 7%, 8%, 9%, 10%, 11%, 12%, 13%, 14%, 15% or greater. Alternatively, the U content of the intron can exceed the U content of the flanked exons by at least 1%, 2%, 3%, 4%, 5%, 6%, 7%, 8%, 9%, 10%, 11%, 12%, 13%, 14%, 15% or greater. See, Simpson et al. (1996) Plant Molecular Biology 32:1-41; Brown et al. (1998) Annu. Rev. Plant Physiol. Plant Mol. Biol. 49:77-95; Schuler et al. (1998) Plant Pre-mRNA Splicing, Bailey-Serres, J. and Gallie, D. R., eds, 1-19, American Society of Plant Physiologist; which discuss the role of the UA rich sequences in plant introns and their role in splicing.

Any intron can be employed in the methods and compositions of the invention. In specific embodiments, the intron comprises ST-LS1, ADH 1 intron 1 from maize (GenBank Accession No. Ay241178), or ADH intron 6 from maize (GenBank Accession No. X04049; Dennis et al. (1984) Nucleic Acid Research 12:3983-4000).

B. Silencing Elements

The intron employed in the methods and compositions of the invention comprises a silencing element. By “silencing element” is intended a polynucleotide which when expressed in a host cell, is capable of reducing or eliminating the level or expression of a target polynucleotide or the polypeptide encoded thereby. The silencing element employed can reduce or eliminate the expression level of the target sequence by influencing the level of the target RNA transcript or, alternatively, by influencing translation and thereby affecting the level of the encoded polypeptide. Methods to assay for functional silencing elements that are capable of reducing or eliminating the level of a sequence of interest are disclosed elsewhere herein. A single intron employed in the invention can harbor one or more silencing elements.

By “reduces” or “reducing” the expression level of a polynucleotide or a polypeptide encoded thereby is intended to mean, the polynucleotide or polypeptide level of the target sequence is statistically lower than the polynucleotide level or polypeptide level of the same target sequence in an appropriate control plant which is not expressing the silencing element. In particular embodiments of the invention, reducing the polynucleotide level and/or the polypeptide level of the target sequence in a modified plant according to the invention results in less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control plant. Methods to assay for the level of the RNA transcript, the level of the encoded polypeptide, or the activity of the polynucleotide or polypeptide are discussed elsewhere herein.

Silencing elements can include, but are not limited to, a sense suppression element, an antisense suppression element, a miRNA, or a hairpin suppression element.

As used herein, a “sense suppression element” comprises a polynucleotide designed to express an RNA molecule corresponding to at least a part of a target messenger RNA in the “sense” orientation. Expression of the RNA molecule comprising the sense suppression element reduces or eliminates the level of the target polynucleotide or the polypeptide encoded thereby. The polynucleotide comprising the sense suppression element may correspond to all or part of the sequence of the target polynucleotide, all or part of the 5′ and/or 3′ untranslated region of the target polynucleotide, all or part of the coding sequence of the target polynucleotide, or all or part of both the coding sequence and the untranslated regions of the target polynucleotide.

Typically, a sense suppression element has substantial sequence identity to the target polynucleotide, optimally greater than about 65% sequence identity, more optimally greater than about 85% sequence identity, about 90%, 91%, 92%, 93%, 94%, 95%, 96%, 97%, 98% or 99% sequence identity. See, U.S. Pat. Nos. 5,283,184 and 5,034,323; herein incorporated by reference. The sense suppression element can be any length so long as it does not interfere with intron splicing and allows for the suppression of the targeted sequence. The sense suppression element can be, for example, 15, 30, 50, 100, 150, 200, 250, 300, 350, 400, 450, 500, 600, 700, 900 or longer.

As used herein, an “antisense suppression element” comprises a polynucleotide which is designed to express an RNA molecule complementary to all or part of a target messenger RNA. Expression of the antisense RNA suppression element reduces or eliminates the level of the target polynucleotide. The polynucleotide for use in antisense suppression may correspond to all or part of the complement of the sequence encoding the target polynucleotide, all or part of the complement of the 5′ and/or 3′ untranslated region of the target polynucleotide, all or part of the complement of the coding sequence of the target polynucleotide, or all or part of the complement of both the coding sequence and the untranslated regions of the target polynucleotide. In addition, the antisense suppression element may be fully complementary (i.e., 100% identical to the complement of the target sequence) or partially complementary (i.e., less than 100% identical to the complement of the target sequence) to the target polynucleotide. In specific embodiments, the antisense suppression element comprises at least 85%, 90%, 91%, 92%, 93%, 94%, 95%, 96%, 97%, 98%, or 99% sequence identity to the target polynucleotide. Antisense suppression may be used to inhibit the expression of multiple proteins in the same plant. See, for example, U.S. Pat. No. 5,942,657. Furthermore, the antisense suppression element can be complementary to a portion of the target polynucleotide. Generally, sequences of at least 25, 50, 100, 200, 300, 400, 450 nucleotides or greater may be used. Methods for using antisense suppression to inhibit the expression of endogenous genes in plants are described, for example, in Liu et al (2002) Plant Physiol. 129:1732-1743 and U.S. Pat. Nos. 5,759,829 and 5,942,657, each of which is herein incorporated by reference.

In specific embodiments, the hairpin suppression element employed in the methods and compositions of the invention comprises in the following order, a first segment, a second segment, and a third segment, where the first and the third segment share sufficient complementarity to allow the transcribed RNA to form a double-stranded stem-loop structure.

The “second segment” of the hairpin comprises a “loop” or a “loop region.” These terms are used synonymously herein and are to be construed broadly to comprise any nucleotide sequence that confers enough flexibility to allow self-pairing to occur between complementary regions of a polynucleotide (i.e., segments 1 and 2 which form the stem of the hairpin). For example, in some embodiments, the loop region may be substantially single stranded and act as a spacer between the self-complementary regions of the hairpin stem-loop. In some embodiments, the loop region can comprise a random or nonsense nucleotide sequence and thus not share sequence identity to a target polynucleotide. In other embodiments, the loop region comprises a sense or an antisense RNA sequence or fragment thereof that shares identity to a target polynucleotide. See, for example, International Patent Publication No. WO 02/00904, herein incorporated by reference. In specific embodiments, the loop region can be optimized to be as short as possible while still providing enough intramolecular flexibility to allow the formation of the base-paired stem region. Accordingly, the loop sequence is generally less than 1000, 900, 800, 700, 600, 500, 400, 300, 200, 100, 50, 25, 20, 15, 10 nucleotides or less.

The “first” and the “third” segment of the hairpin RNA molecule comprise the base-paired stem of the hairpin structure. The first and the third segments are inverted repeats of one another and share sufficient complementarity to allow the formation of the base-paired stem region. In specific embodiments, the first and the third segments are fully complementary to one another. Alternatively, the first and the third segment may be partially complementary to each other so long as they are capable of hybridizing to one another to form a base-paired stem region. The amount of complementarity between the first and the third segment can be calculated as a percentage of the entire segment. Thus, the first and the third segment of the hairpin RNA generally share at least 50%, 60%, 70%, 80%, 85%, 90%, 91%, 92%, 93%, 94%, 95%, 96%, 97%, 98%, 99%, up to and including 100% complementarity.

The first and the third segment are at least about 1000, 500, 400, 300, 200, 100, 50, 40, 30, 25, 20, 15 or 10 nucleotides in length. In specific embodiments, the length of the first and/or the third segment is about 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 20 nucleotides. In other embodiments, the length of the first and/or the third segment comprises at least 10-20 nucleotides, 20-35 nucleotides, 30-45 nucleotides, 40-50 nucleotides, 50-100 nucleotides, or 100-300 nucleotides. See, for example, International Publication No. WO 0200904. In specific embodiments, the first and the third segment comprises at least 20 nucleotides having at least 85% complementary to the first segment. In still other embodiments, the first and the third segments which form the stem-loop structure of the hairpin comprises 3′ or 5′ overhang regions having unpaired nucleotide residues.

In specific embodiments, the sequences used in the first, the second, and/or the third segments comprise domains that are designed to have sufficient sequence identity to a target polynucleotide of interest and thereby have the ability to decrease the level of expression of the target polynucleotide. The specificity of the inhibitory RNA transcripts is therefore generally conferred by these domains of the silencing element. Thus, in some embodiments of the invention, the first, second and/or third segment of the silencing element comprise a domain having at least 10, at least 15, at least 19, at least 20, at least 21, at least 22, at least 23, at least 24, at least 25, at least 30, at least 40, at least 50, at least 100, at least 200, at least 300, at least 500, at least 1000, or more than 1000 nucleotides that share sufficient sequence identity to the target polynucleotide to allow for a decrease in expression levels of the target polynucleotide when expressed in an appropriate cell. In other embodiments, the domain is between about 15 to 50 nucleotides, about 20-35 nucleotides, about 25-50 nucleotides, about 20 to 75 nucleotides, about 40-90 nucleotides about 15-100 nucleotides.

In specific embodiments, the domain of the first, the second, and/or the third segment has 100% sequence identity to the target polynucleotide. In other embodiments, the domain of the first, the second and/or the third segment having homology to the target polypeptide have at least 50%, 60%, 70%, 80%, 85%, 90%, 91%, 92%, 93%, 94%, 95%, 96%, 97%, 98%, 99%, or greater sequence identity to a region of the target polynucleotide. The sequence identity of the domains of the first, the second and/or the third segments to the target polynucleotide need only be sufficient to decrease expression of the target polynucleotide of interest. See, for example, Chuang and Meyerowitz (2000) Proc. Natl. Acad Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965; each of which is herein incorporated by reference.

The amount of complementarity shared between the first, second, and/or third segment and the target polynucleotide or the amount of complementarity shared between the first segment and the third segment (i.e., the stem of the hairpin structure) may vary depending on the organism in which gene expression is to be controlled. Some organisms or cell types may require exact pairing or 100% identity, while other organisms or cell types may tolerate some mismatching. In some cells, for example, a single nucleotide mismatch in the targeting sequence abrogates the ability to suppress gene expression. In these cells, the suppression cassettes of the invention can be used to target the suppression of mutant genes, for example, oncogenes whose transcripts comprise point mutations and therefore they can be specifically targeted using the methods and compositions of the invention without altering the expression of the remaining wild-type allele.

Any region of the target polynucleotide can be used to design the domain of the silencing element that shares sufficient sequence identity to allow expression of the hairpin transcript to decrease the level of the target polynucleotide. For instance, the domain can be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof. In some instances to optimize the siRNA sequences employed in the hairpin, the synthetic oligodeoxyribonucleotide/RNAse H method can be used to determine sites on the target mRNA that are in a conformation that is susceptible to RNA silencing. See, for example, Vickers et al. (2003) J. Biol. Chem 278:7108-7118 and Yang et al. (2002) Proc. Natl. Acad. Sci. USA 99:9442-9447, herein incorporated by reference. These studies indicate that there is a significant correlation between the RNase-H-sensitive sites and sites that promote efficient siRNA-directed mRNA degradation.

It is recognized that multiple members of a gene family can be targeted using this method. For example, a silencing element can be designed, based on sequence identity shared among various members of a gene family, and thereby decrease the expression of multiple related polynucleotides. Alignment of the family members can be used to design such a silencing element.

It is further recognized that multiple unrelated target polynucleotides can also be targeted. For example, where the purpose is to decrease the level of expression of more than one target polynucleotide, regions of DNA whose sequence corresponds to that present in the different target polynucleotides can be combined into the first, second, and/or third segment of the silencing element. In this manner, the suppression cassette is designed to express a single fusion RNA transcript having specificity for multiple target polynucleotides.

In some embodiments, the second segment (i.e., the loop region) may comprise all or part of a sequence corresponding to a target polynucleotide of interest. While the stem structure (i.e., the first and third segment) of the hairpin transcript will, in most instances, be designed to target a gene product, it is contemplated that the base-paired stem structure of the inhibitory RNA transcript may be formed by the hybridization of a first segment and a second segment, neither of which correspond to an endogenous sequence found in the organism of interest. In this embodiment, it is the loop region that determines the specificity of the RNA interference. See, for example, WO 02/00904, herein incorporated by reference.

In addition, transcriptional gene silencing (TGS) may be accomplished through use of a hairpin suppression element where the inverted repeat of the hairpin shares sequence identity with the promoter region of a target polynucleotide to be silenced. See, for example, Aufsatz et al. (2002) PNAS 99 (Suppl. 4):16499-16506 and Mette et al. (2000) EMBO J 19(19):5194-5201.

In other embodiments, the silencing element contained in the intron sequence could comprise a micro RNA (miRNA). miRNAs are regulatory agents consisting of about 22 ribonucleotides which are highly efficient at inhibiting the expression of endogenous genes. See, for example Javier et al. (2003) Nature 425: 257-263, herein incorporated by reference. For miRNA interference, the silencing element is designed to express an RNA molecule that forms a hairpin structure containing a 22-nucleotide sequence that is complementary to a target polynucleotide of interest. Specifically, the miRNA can comprise 22 nucleotides of the sequence having homology to a target polynucleotide in sense orientation and 21 nucleotides of a corresponding antisense sequence that is complementary to the sense sequence. miRNA molecules are highly efficient at inhibiting the expression of the target polynucleotides, and the RNA interference they induce is inherited by subsequent generations of plants.

The intron employed in the compositions and methods can also contain a translation stop codon in the 5′ proximal region, which if present in a cytoplasmic mRNA, would signal the diversion of the defective pre-mRNA to the NMD pathway.

The silencing element can be designed to reduce or eliminate the level of any polynucleotide of interest or the polypeptide encoded thereby. In specific embodiments, the polynucleotide of interest or the polypeptide encoded thereby that is targeted for suppression is an agronomically important sequence. By “agronomically important sequence” is intended any polypeptide or polynucleotide, which when expressed produces an agronomically important trait. An agronomically important trait includes any phenotype in a plant that is useful or advantageous for food and/or feed production or food and/or feed products. Non-food agricultural products such as paper products are also included.

A partial list of agronomically important polynucleotide include sequences that promote the following traits in a plant or plant part: a modulation in the fatty acid composition in a plant, a modulation in the pathogen defense mechanism, a modulation in oil production (including an increase in oleic acid levels, a modulation in saturated and/or unsaturated oils), a modulation in tocol content, an increase in the level of lysine and sulfur, an increase in the quality and/or quantity of essential amino acids, a modulation in starch synthesis, a modulation in phytic acid levels, a modulation in pest resistance, a modulation in disease resistance, a modulation in herbicide resistance, a modulation of male sterility phenotypes, a modulation in plant vigor, a modulation in development time (i.e., time to harvest), an enhanced nutrient content, an enhanced yield, a novel growth pattern, an improved digestibility and energy value of the grain, a modulation in hemicellulose, a modulation in cellulose production, a modulation of salt, heat, drought and/or cold tolerance. In specific embodiments, an agronomically important trait includes a modulation of tocol content, oleic acid content, phytic acid content, or amino acid composition. Agronomically important sequences do not include reporter or marker genes, such as, GFP, luciferase, or lacZ.

Set forth below are non-limiting silencing elements that can be employed in the compositions and methods of the invention to reduce or eliminate the level of an agronomically important polynucleotide or polypeptide encoded thereby. The silencing elements employed herein are designated herein by the name of the target gene product.

(i) ADR-Glucose Pyrophosphorylase (AGP) Silencing Elements

An “AGP silencing element” refers to a silencing element that is capable of reducing or eliminating the level (i.e., inhibiting the expression) of an ADP-glucose pyrophosphorylase (AGP) at the level of transcription and/or translation.

Various AGP-like polypeptides (native or biologically active variants or fragments thereof) are known and can be targeted for suppression using the AGP silencing element of the invention. AGP catalyzes the first committed reaction in the pathway of starch synthesis. Numerous genes encoding the small and large subunits of AGP from plants have been described. See, for example, Smith-White et al. (1992) J. Mol. Evol. 34: 449-464, herein incorporated herein by reference in its entirety. The corresponding genes identified from maize are the endosperm specific Bt2 (GenBank Acc. No. AF334959) and Sh2 (GenBank Acc. No. AF334959) genes. See, for example, Bae et al. (1990) Maydica 35: 317-322; Bhave et al. (1990) Plant Cell 2 581-588; Deyner et al. (1996) Plant Physiology 112, 2 779-785; each of which is incorporated herein in its entirety by reference. Additional AGP polynucleotides include AGP1 and AGP2. AGP1 represents the large subunit of the embryo isoform, whereas AGP2 corresponds to the small subunit. See, for example, Giroux et al. (1995) Plant Physiol. 108: 1333-1334, U.S. Pat. No. 6,232,529, Hannah et. al. (2001) Plant Physiol 127: 173-183, U.S. patent application Ser. No. 11/021,666, and, Harvengt et al. (1996) Plant Physiol. 112: 1399 (Accession No. X96771), each of which is herein incorporated by reference. The silencing element of the invention can be designed to reduce or eliminate expression of a native AGP2 sequence. Alternatively, the silencing element can be designed to reduce or eliminate expression of a sequence encoding a biologically active variant or fragment of AGP2.

Reducing or eliminating the level of at least one AGP polypeptide or a biologically active variant or fragment thereof in the cell, plastids, and/or the cytoplasm will disrupt starch biosynthesis and/or enhance oil production. See, Stark et al. (1992) Science 258:287-292 and U.S. Pat. No. 6,262,529. By “disrupting starch biosynthesis” is intended a modification to the starch anabolic pathway that results in a net decrease in starch production when compared to a control plant. By “disrupting storage of starch” is intended any modification to the starch catabolic pathway that results in an increase in starch degradation and net decrease in starch accumulation.

A decrease in the activity of an AGP-like polypeptide or a biologically active variant or fragment thereof, such as AGP1 and AGP2, can be measured by assaying for the activity of ADP-glucose pyrophosphoylase directly (EC 2.7.7.27). Briefly, the activity is measured as described in Singletary et al. (1980) Plant Physiol. 92: 160-167, herein incorporated in its entirety by reference. Alternatively, the level of the polypeptide or the transcript can be assayed by Western analysis or Northern analysis, respectively. In still another embodiment, the level is determined by assaying for the desired suppression phenotype.

(ii) A Fatty Acid Desaturase Silencing (FAD)Elements

A “FAD silencing element” refers to a silencing element that is capable of reducing or eliminating the level (i.e., inhibiting the expression) of a fatty acid desaturase (FAD), for example, at the level of transcription and/or translation. One or more of the fatty acid desaturases can be targeted by the silencing element.

Various FADs (native or biologically active variants thereof) are known and can be targeted for suppression using the FAD silencing element of the invention. FADs of interest include, stearoyl-acyl-carrier-protein desaturase (Fad1; see U.S. Pat. No. 6,117,677), delta-15 desaturase (omega-3) (Fad3; Shah et al. (1997) Plant Physiol. 11:1533-1539), delta-4 (trans) desaturase (Fad4; Xiao et al. (2001) J. Biol. Chem. 276:31561-31566), delta-7 desaturase, (Fad5; see U.S. Pat. No. 6,635,451), omega-6 fatty-acid desaturase (Fad6; see U.S. Pat. No. 6,635,451), omega-3 fatty-acid desaturase (Fad7; Iba et al. (1993) J. Biol. Chem. 268:24099-24105), delta-5 desaturase (see U.S. Pat. No. 6,589,767), delta-9-desaturase (see U.S. Pat. No. 5,723,595), fatty acyl-CoA:fatty alcohol acyltransferase (wax synthase; see U.S. Pat. No. 6,492,509), beta-ketoacyl-ACP synthase in an antisense or sense orientation (see U.S. Pat. No. 6,483,008), and delta-12 fatty acid desaturase (FAD2), an enzyme that converts oleic acid to linoleic acid by introducing a double bond at the delta-12 position (Okuley et al. (1994) Plant Cell 6:147-58).

Various native FAD2 sequences or a biologically active variant or fragment thereof can be targeted for suppression by the compositions and methods of the invention. See, for example, GenBank Accession No. NM_{—}112047; GenBank Accession No. AF243045; European Patent No. EP0668919 B1; U.S. Pat. No. 6,291,742; U.S. Pat. No. 6,310,194; U.S. Pat. No. 6,323,392; U.S. Pat. No. 6,372,965; U.S. Patent Application Publication No. 20030033633; and U.S. Patent Application Publication No. 20030140372; all of which are incorporated in their entirety herein by reference. In maize, two FAD2 proteins have been identified: zmFAD2-1 and zmFAD2-2 (U.S. application Ser. No. 11/021,666, Kinney et al. (2001) Biochem. Soc. Trans. 30:1099-1103; and Mikkilineni et al. (2003) Theor. Appl. Genet. 106:1326-1332), each of which is herein incorporated by reference. The silencing element of the invention can be designed to reduce or eliminate expression of the FAD2 sequence. Alternatively, the silencing element can be designed to reduce or eliminate expression of a sequence encoding a biologically active variant or fragment of FAD2.

Reducing or eliminating the level of at least one FAD polypeptide or a biologically active variant or fragment thereof in the cell, plastids, and/or the cytoplasm will modify the oil characteristics of the plant. FAD2, or a biologically active variant or fragment thereof, converts the delta-12 single bond of oleic acid (C18:1) into a conjugated double bond, thus producing linoleic acid (C18:2). Therefore, inhibiting the expression or function of FAD2 or a biologically active variant thereof prevents the conversion of oleic acid into linoleic acid, and thus, oleic acid accumulates in the plant or plant part thereof and the level of linoleic acid is decreased. Methods to assay for oleic acid and linoleic acid levels are known in the art. See, for example, U.S. application Ser. No. 11/021,666. Using the methods and compositions disclosed herein, total oil production can be increased and/or the characteristics of the oil can be modified. As used herein, a modulation of oleic acid levels comprises any increase or decrease in oleic acid content when compared to a control plant or plant part. In one embodiment, the oleic acid level is decreased or increased by 1%, 5%, 10%, 20%, 30%, 40%, 50%, 60%, 70%, 80%, 90%, or greater.

(iii) Phytic Acid Silencing Elements

A “phytic acid silencing element” refers to a silencing element that is capable of reducing or eliminating the level (i.e., inhibiting the expression) of a sequence involved in phytic acid biosynthesis.

Various sequence involved in phytic acid biosynthesis are known and can be targeted for suppression using the phytic acid silencing element of the invention. “Phytic acid”, as used herein means myo-inositol tetraphosphoric acid, myo-inositol pentaphosphoric acid, or myo-inositol hexaphosphoric acid. As a salt with cations, phytic acid is “phytate.” Phytic acid biosynthesis sequences (native or biologically active variants thereof) which can be targeted for suppression include, but are not limited to, LPA1 (U.S. application Ser. No. 11/133,075), LPA2 (U.S. Publication No. 20050202486 and U.S. Publication No. 20030079247); LPA3 (U.S. application Ser. No. 11/132,864); myo-inositol 1-phosphate synthase (MI1PS), inositol 1,3,4-trisphosphate 5/6 kinases (ITPKs) and myo-inositol monophophatase (IMP) (see WO 99/05298) and the like, the disclosures of which are herein incorporated by reference. See also, U.S. Pat. No. 6,855,869, herein incorporated by reference.

In specific embodiments, reducing or eliminating the expression of a phytic acid biosynthesis polypeptide results in a decrease in the level of phytic acid in the plant or plant part. For example, one or more of a Low Phytic Acid 1 (LPA1) silencing element, a Low Phytic Acid 2 (LPA2) silencing element, or a Low Phytic Acid 3 (LPA3) silencing element can be used to target suppression of LPA1, LPA2, and LPA3 or a biologically active variant or fragment thereof. See, for example, U.S. application Ser. No. 11/133,075; U.S. application Ser. No. 11/132,864; U.S. Pat. No. 5,689,054 and U.S. Pat. No. 6,111,168, each of which is herein incorporated by reference. The silencing element of the invention can be designed to reduce or eliminate expression of the LPA1, LPA2, and/or LPA3 sequence. Alternatively, the silencing element can be designed to reduce or eliminate expression of a sequence encoding a biologically active variant or fragment of LPA1, LPA2, or LPA3.

Methods to assay for the level of phytic acid in a plant are known in the art. See, for example, U.S. Pat. No. 6,111,168 and U.S. Application Publication 20030009011, both of which are herein incorporated by reference. As used herein, a modulation of phytic acid content comprises any increase or decrease in phytic acid level when compared to a control plant or plant part. In one embodiment, the phytic acid level is decreased by 1%, 5%, 10%, 20%, 30%, 40%, 50%, 60%, 70%, 80%, 90%, or greater.

(iv) Prolamin Silencing Elements

A “silencing element for a prolamin polypeptide” refers to a silencing element that is capable of reducing or eliminating the level (i.e., inhibiting the expression of) a prolamin polypeptide (native or biologically active variants thereof) at the level of transcription and/or translation.

Various prolamin polypeptides (native or biologically active variants thereof) are known and can be targeted for suppression using the prolamin silencing element of the invention. Prolamins are the major endosperm storage protein of all cereal grains. The complete amino acid sequence of many prolamin polypeptides are know and has allowed the structure and properties of the prolamin superfamily to be characterized. See, Shewry et al. (1990) Biochem Journal 267:1-12 and Shewry et al. (2002) Journal of Experimental Botany 53:947-958. The prolamin polypeptides of maize (called zeins) are classified into the following classes: α-zeins, β-zeins, γ-zeins, and δ-zeins. See, Coleman et al. (1999) Seed Proteins Dordrecht: Kluwer Academic Publishers 109-139 and Leite et al. (1999) Seed Proteins Dordrecht: Kluwer Academic Publishers 141-157.

Any α-zein sequence can be targeted by an appropriate a-zein silencing element including, for example, a sequence encoding the 19K α-zein polypeptide or a biologically active variant or fragment thereof and a sequence encoding the 22K α-zein polypeptide or a biologically active variant or fragment thereof. See, for example, Segral et al. (2003) Genetics 165:387-397, GenBank Accession No. X61085, AF371277 and X55661, and Kim et al. (2004) Plant Physiol. 134 (1), 380-387; herein incorporated by reference. Any γ-zein sequence can be targeted by an appropriate y-zein silencing element including, for example, sequence encoding the 50 kD γ-zein polypeptide (GenBank Acc. No. AF371263 and U.S. Pat. No. 6,858,778), the 16 kD γ-zein polypeptide (GenBank Acc. No. AF371261), or a biologically active variant or fragment thereof. Any δ-zein sequence can be targeted by a δ-zein silencing element including, for example, a polynucleotide encoding the 10 kD δ-zein polypeptide (GenBank Accession No. AF371266) and the 18 kD δ-zein polypeptide (GenBank Acc. No. AF371265), or a biologically active variant or fragment thereof.

In other embodiments, a lysine-ketoglutarate reductase (LKR) (EC 1.5.1.8) is targeted by an appropriate LKR silencing element. LKR is the first enzyme in the Lys catabolism pathway (also named as Lys 2-oxoglutarate reductase), which condenses Lys and alpha-ketoglutarate into saccharopine and uses the co-factor NADPH. The nucleotide and amino acid sequence of many LKR polypeptides are known. See, for example, Miron et al. (2000) Plant Physiology 123:655-663, Kemper et al. (1999) The Plant Cell 11:1981-1993, and, Epelbaum et al. (1997) Plant Molecular Biology 35:735-748, each of which is herein incorporated by reference.

The silencing element of the invention can be designed to reduce or eliminate expression of the 27 kD gamma zein, the 50 kD gamma zein, or the LKR sequence. Alternatively, the silencing element can be designed to reduce or eliminate expression of a sequence encoding a biologically active variant or fragment of 27 kD gamma zein, 50 kD gamma zein, or LKR.

Decreasing or eliminating the level of at least one prolamin polypeptide and/or an LKR polypeptide can, for example, improve the amino acid composition/nutrient value of the seed, improve digestibility and nutrient availability, improve response to feed processing, improve silage quality, and increase efficiency of the wet or dry milling process. See, for example, U.S. Pat. No. 6,858,778, herein incorporated by reference, for assays to measure these various qualities. Alternatively, the level of the polypeptide or the transcript can be assayed by Western analysis or Northern analysis, respectively.

In specific embodiments, a γ-zein gene is suppressed to increase the nutritional value of seed, particularly by increasing the lysine content of the seed, and the digestibility of seed. Increases in the lysine content of such seed can be at least about 10%, 15%, 20%, 25%, 30%, 35%, 40%, 45%, and 50% or higher. Digestibility can be improved by at least 3%, 6%, 9%, 12%, 15%, 20% and greater. See, for example, U.S. Pat. No. 6,858,778, herein incorporated by reference, for assays to measure these various qualities.

(C) Overexpressed Polynucleotides

The polynucleotide which harbors the intron of the invention comprises any polynucleotide sequence, which when expressed in a host cell and upon the splicing of the intron, will result in an increased level of a polynucleotide of interest when compared to an appropriate control.

By “increases” or “increasing” the level of a polynucleotide is intended to mean, the polynucleotide level is statistically higher than the polynucleotide level of the same sequence in an appropriate control plant. In particular embodiments of the invention, increasing the polynucleotide level in a modified plant according to the invention results in at least about a 1%, 5%, 10%, 20%, 30%, 50%, 60%, 70%, 90%, 100%, 150% or greater increase in the polynucleotide level of the same sequence in an appropriate control plant. Methods to assay for the level of the RNA transcript, the level of the encoded polypeptide, or the activity of the polynucleotide or polypeptide are discussed elsewhere herein.

The intron of the invention can be located anywhere in the overexpressed polynucleotide sequence, so long as the position allows for the intron to be spliced from the transcript and the flanking exons are rejoined in a manner that allows for the expression of the overexpressed polynucleotide. For example, the intron can be found in the coding region, the 5′ untranslated region, or the 3′ untranslated region of the overexpressed sequence. The overexpressed polynucleotide can comprise 1, 2, 3, 4 or more introns, each harboring the same or a different silencing element.

In specific embodiments, the polynucleotide employed in the invention comprises a marker gene or a reporter gene. In this embodiment, the marker or reporter sequence provides an assay that allows the splicing of the intron comprising the silencing element to be monitored. In other embodiments, the polynucleotide of interest that is overexpressed is an agronomically important sequence. As discussed above, a variety of agronomically important sequences can be employed as the overexpressed polynucleotide. Non-limiting examples of such sequences are set forth below.

(i) Tocol Biosynthesis Sequences

In one embodiment, the recombinant polynucleotide of the invention, when expressed in a plant or plant part, modulates tocol content in a plant. As used herein, a “polypeptide involved in tocol biosynthesis” comprises any polypeptide which is involved, either directly or indirectly, in modulating tocol content in a plant. The term “tocol” refers generally to any of the tocopherol and tocotrienol molecular species that are known to occur in biological systems. Such compounds comprise a series of related benzopyranols (or methyl tocols) including tocopherols and tocotrienols. Tocopherols have a saturated C16 side chain, and the tocotrienols have an unsaturated C16 side chain with three double bonds. The four main constituents of tocols are termed alpha, beta, gamma and delta. See, for example, IUPAC-IUB JCBN (1982) Arch. Biochem. Biophys. 218: 347-348; IUPAC-IUB JCBN (1982) Eur. J. Biochem. 123: 473-475; IUPAC-IUB JCBN (1982) Mol. Cell. Biochem. 49: 183-185; Liébecq (1982) Pure Appl. Chem. 54: 1507-1510; Biochemical Nomenclature and Related Documents (1992) 2nd edition, Portland Press: 239-241, each of which is herein incorporated by reference.

The term “tocol level” refers to the total amount of tocopherol and tocotrienol in a whole plant, plant part, plant tissue, or plant cell or in a microbial host. The term “tocol composition” refers both to the ratio of the various tocols produced in any given biological system and to altered characteristics, such as antioxidant activity, of any one tocol compound.

“Modulating tocol content” includes any decrease or increase in the total tocol level and/or the tocol composition in a whole plant, plant part, plant tissue, plant cell or microbial host. For example, modulating tocol content can comprise either an increase or a decrease in overall tocol level of about 0.1%, 0.5%, 1%, 5%, 10%, 20%, 30%, 40%, 50%, 60%, 70%, 80%, 90%, 100%, 120% or greater when compared to a control plant or plant part. Alternatively, the modulated tocol level can include about a 0.5 fold, 1 fold, 2 fold, 4 fold, 8 fold, 16 fold, 32 fold or greater overall increase or decrease in tocol level in the plant or the plant part when compared to a control plant or plant part.

Moreover, the modulation of the tocol content can also include a modulation in tocol composition: a change in the ratio of one or more tocols and/or the altered characteristic of one or more tocol. For example, the ratio of various tocols such as the alpha, beta, gamma and/or delta tocotrienols and/or tocopherols could be altered and thereby modulate the tocol content of the plant or plant part when compared to a control plant. Similarly, the tocotrienol content (i.e., tocotrienol composition and/or level) or the tocopherol content (i.e., tocopherol composition and/or level) can be modulated as outlined above.

Methods for assaying for a modulation in tocol content, tocopherol content and/or tocotrienol content are known in the art. For example, representative methods to measure tocol content, such as, extraction, immunopurification, chromatographic separation, gas chromatography-mass spectrometry, and quantification by ELISA methods can be found in, for example, Kamal-Eldi et al. (2000) J Chromatogr A 881:217-227; Bonvehi et al. (2000) J. AOAC Intl. 83:627-634; Goffman et al. (2001) J Agric. Food Chem. 49:4990-4994; Abidi (2000) J Chromatogr A 881:197-216; Gomez-Coronado et al. (2003) J. Agric Food Chem 51:5196-201; Panfili et al. (2003) J Agric Food Chem 51:3940-4; Huo et al. (1999) J Chromatogr B Biomed Sci Appl 724:249-55; U.S. Application Publication 20020042527; U.S. Pat. No. 5,908,940; U.S. Application Publication 2004/0034886; and, Frega et al. (1998) J. Amer. Oil Chem. Soc. 75:1723-1728. Each of these references is herein incorporated by reference.

Methods to assay for the activity of tocols are also known. For example, lipophilic antioxidant activity of tocols may be measured by various assays including the inhibition of the coupled auto-oxidation of linoleic acid and β-carotene and oxygen radical absorbance capacity (ORAC). See, Serbinova et al. (1994) Meth. Enzymol. 234:354-366; Emmons et al. (1999) J. Agric. Food Chem. 47:4894-4898); and, Huang et al. (2002) J. Agric. Food Chem. 52:2993-7. See also, Andarwulan et al. (1999) J Agric Food Chem 47:3158-63 and Fukuzawa et al. (1982) Lipids 17:511-3.

Such polypeptides include, but are not limited to, y-tocopherol methyltransferase (U.S. Pat. No. 6,642,434, WO 99/04622, and Shintani et al. (1998) Science 282:2098-2100); p-hydroxyphenylpyruvate dioxygenase (HPPDase) (WO 97/27285, Garcia et al. (1997) Biochem J. 325:761 and Norris et al. (1998) Plant Physiol. 117:1317); tocopherol cyclase (U.S. Pat. No. 6,872,815 and Kanwisher et al. (2005) Plant Physiol. 137:713-723); 1-deoxy-D-xylose-5-phosphate synthase (WO 00/08169); geranylgeranyl-pyrophosphate oxidoreductase (WO 00/08169); tyrosine amino transferase (US Application Publication 20040086989); geranylgeranyl reductase (GGR) (WO 99/23231); homogentisate geranylgeranyl transferases (HGGT) (U.S. Patent Publication 2004/0034886); and, homogentisate phytyltransferase (HPT) (U.S. Pat. No. 6,787,683); each of these references is herein incorporated by reference.

In one embodiment of the invention, a homogentisate geranylgeranyl transferases (HGGT) polypeptide (native or biologically active variants thereof) is overexpressed using the recombinant polynucleotide. The HGGT polypeptide family comprises polypeptides that catalyze the condensation of homogentisate (or homogentisic acid) and geranylgeranyl pyrophosphate (or geranylgeranyl diphosphate). This reaction is an important step in tocotrienol biosynthesis and can result in the modulation of the tocol content.

HGGT polypeptides are members of the UbiA prenyltransferase family. Members of this family are distinguished by the presence of a UbiA consensus motif. Of the known members of this family, HGGTs are most closely related to HPTs. Using amino acid sequence alignments, one skilled in the art can distinguish HGGT polypeptides from HPT polypeptides, other members of the UbiA prenyltransferase family. See U.S. Patent Application No. 2004/0034886 for conserved amino acid residue of HGGT and characterized HGGT-specific motifs. As outlined in detail elsewhere herein, biologically active variants and fragments of the HGGT polynucleotide and polypeptide can also be employed in the methods of the invention. Such variants and fragments are known in the art. See, for example, U.S. Application Publication 2004/0034886, which is herein incorporated by reference.

Biologically active fragments and variants of a HGGT polypeptide will continue to retain HGGT activity. As used herein, “HGGT activity” is defined as the ability of a polypeptide to catalyzes the condensation of homogentisate (or homogentisic acid) and geranylgeranyl pyrophosphate (or geranylgeranyl diphosphate). Various methods are known in the art to assay for this activity. For example, the HGGT polypeptide can be expressed in a dicot which does not produce tocotrienols. Such a system includes tobacco callus which is enriched in tocopherol. Expression of an HGGT polypeptide in this system will increase accumulation of tocotrienols compared to the appropriate control plant or plant part. See, for example, Cahoon et al. (2003) Nature Biotechnology 21:1082-1087, and, US Application Publication 2004/0034886, herein incorporated by reference.

(ii) Barley High Lysine (BHL) Polypeptides

In one embodiment of the invention, the overexpressed polynucleotide of the recombinant polynucleotide comprises a polypeptide that is capable of modulating the amino acid composition of the plant. By “modulating amino acid composition” is intended to mean any increase or decrease in the total amino acid level in a plant or plant part and/or any increase or decrease in the level of any individual amino acid in a plant or plant part. The polypeptide can be a modified protease polypeptide, and in specific embodiments, it is a modified chymotrypsin inhibitor. As used herein, “Barley High Lysine polypeptides” or “BHL polypeptides” are nutritionally enhanced derivatives of barley chymotrypsin inhibitor-2 (CI-2). Such polynucleotides have an enriched content of essential amino acids, such as, lysine, methionine, tryptophan, threonine, isoleucine etc. By “derivative” is intended that the chymotrypsin inhibitor polypeptide may be truncated, modified (internal and/or terminal substitutions and/or deletions). Such polypeptides are known in the art and include, for example, BHL1, BHL2, BHL3, BHL4, BHL5, BHL6, BHL7, BHL8, and BHL9. Such sequences can be found, for example, in U.S. Pat. No. 6,800,726; Rao et al. (2001) J. Agric. Food Chem. 49:3443-3451; Roesler et al. (2000) Protein Sci 9:1642-1650; and Roesler et al. (1999) Protein Eng. 12:967-973, each of which is herein incorporated by reference.

As outlined in detail elsewhere herein, biologically active variants and fragments of a BHL polynucleotide and polypeptide can also be employed in the methods of the invention. Such variants and fragments are known in the art. See, for example, U.S. Pat. No. 6,800,726; Rao et al. (2001) J. Agric. Food Chem. 49:3443-3451; Roesler et al. (2000) Protein Sci 9:1642-1650; and Roesler et al. (1999) Protein Eng. 12:967-973. BHL polynucleotides or biologically active variants or fragments thereof have an increased percentage of essential amino acids and are readily digestible by enzymes of the gastrointestinal track. See, Roesler et al. (2001) J. Agri. Food Chem. 49:3443-3431 for a discussion of various assays. The recombinant polynucleotide of the invention can comprise a BHL9 sequence which is overexpressed in the plant or plant part. Alternatively, the recombinant polynucleotide of the invention can comprise a biologically active variant or fragment of BHL9.

(D) Variants and Fragments

By “fragment” is intended a portion of the polynucleotide or a portion of the amino acid sequence and hence protein encoded thereby. Fragments of a polynucleotide may encode protein fragments that retain the biological activity of the native protein. Alternatively, fragments of a polynucleotide that are useful as a silencing element do not need to encode fragment proteins that retain biological activity. Thus, fragments of a nucleotide sequence may range from at least about 10, about 15, 20 nucleotides, about 50 nucleotides, about 75 nucleotides, about 100 nucleotides, 200 nucleotides, 300 nucleotides, 400 nucleotides, 500 nucleotides, 600 nucleotides, 700 nucleotides and up to the full-length polynucleotide employed in the invention. Methods to assay for the activity of a desired silencing element or for the overepxressed polynucleotide and/or polypeptide are described elsewhere herein.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the polypeptides employed in the invention. Variant polynucleotides also include synthetically derived polynucleotide, such as those generated, for example, by using site-directed mutagenesis, but continue to retain the desired activity. Generally, variants of a particular polynucleotide of the invention (i.e., an intron, an overexpressed polynucleotide, or a silencing element) will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides employed in the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present invention are biologically active, that is they continue to possess the desired biological activity of the native protein, as discussed elsewhere herein. Such variants may result from, for example, genetic polymorphism or from human manipulation. Biologically active variants of a native protein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(E) Expression Cassettes

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

Various modifications can be made to the recombinant polynucleotide of the invention. For example, the recombinant polynucleotide can comprise multiple introns with the same or different silencing elements. In addition, multiple silencing elements can be contained in the same intron and/or the recombinant polynucleotide can comprise more than one polynucleotide for overexpression.

The recombinant polynucleotide of the invention can be provided in an expression cassette for expression in the plant of interest. The cassette will include 5′ and 3′ regulatory sequences operably linked to the polynucleotide construct of the invention. “Operably linked” is intended to mean a functional linkage between two or more elements. For example, an operable linkage between a polynucleotide of interest and a regulatory sequence (i.e., a promoter) is functional link that allows for expression of the polynucleotide of interest. An operably linkage between an intron and a polynucleotide of interest is one that allows the intron to be spliced from the polynucleotide in a manner that allows the polynucleotide to continue to encode the desired polypeptide or retain the desired activity. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide construct to be under the transcriptional regulation of the regulatory regions. The expression cassette may additionally contain selectable marker genes.

The expression cassette will include in the 5′-3′ direction of transcription, a transcriptional and translational initiation region (i.e., a promoter), the recombinant polynucleotide, and a transcriptional and translational termination region (i.e., termination region) functional in a plant. The regulatory regions (i.e., promoters, transcriptional regulatory regions, and translational termination regions), the overexpressed polynucleotide or the intron employed in the invention may be native/analogous to the host cell or to each other. Alternatively, the regulatory regions and/or the recombinant polynucleotide or the intron employed in the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. The intron employed in the invention maybe native to the overexpressed polynucleotide or it may be heterologous to the overexpressed polynucleotide. As used herein, a chimeric gene comprises a coding sequence operably linked to a transcription initiation region that is heterologous to the coding sequence. As used herein, a recombinant polynucleotide is a polynucleotide sequence that has been modified from its native state by human intervention.

The termination region may be native with the transcriptional initiation region, may be native with the operably linked overexpressed polynucleotide, maybe native with the intron, may be native with the plant host, or may be derived from another source (i.e., foreign or heterologous) to the promoter, the polynucleotide of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the recombinant polynucleotide may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picomavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, N.Y.), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention, including the native promoter of the polynucleotide sequence of interest. The promoters can be selected based on the desired outcome. The polynucleotide constructs can be combined with constitutive, tissue-preferred, or other promoters for expression in plants. Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

An inducible promoter, such as a from a pathogen-inducible promoter, could also be employed. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Chemical-regulated promoters can be used to modulate the expression of the polynucleotide construct in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, CimI (cytokinin-induced message); cZ19B1 (maize 19 kDa zein); milps (myo-inositol-1-phosphate synthase) (see WO 00/11177 and U.S. Pat. No. 6,225,529; herein incorporated by reference). Gamma-zein is an endosperm-specific promoter. Globulin 1 (Glb-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed; herein incorporated by reference. Embryo-specific promoters include ESR (U.S. Application Publication 20040210960) and lecl (U.S. patent application Ser. No. 09/718,754, filed Nov. 22, 2000). Additional embryo-specific promoters are disclosed in Sato et al. (1996) Proc. Natl. Acad. Sci. 93:8117-8122; Nakase et al. (1997) Plant J. 12:235-56; and Postma-Haarsma et al. (1999) Plant Mol. Biol. 39:257-71. Endosperm-preferred promoters include epp1 and eep2 as disclosed in U.S. Patent Application Publication 20040237147. Additional endosperm-specific promoters are disclosed in Albani et al. (1985) EMBO 3:1505-15; Albani et al. (1999) Theor. Appl. Gen. 98:1253-62; Albani et al. (1993) Plant J. 5:353-55; Mena et al. (1998) The Plant Journal 116:53-62, and Wu et al. (1998) Plant Cell Physiology 39:885-889. Immature ear tissue-preferred promoters can also be employed.

The expression cassette can also comprise a selectable marker gene or a reporter gene for the selection of transformed cells. Selectable marker genes are utilized for the selection of transformed cells or tissues. Selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad Sci. USA 89:3952-3956; Baim et al. (1991) Proc. Natl. Acad Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad Sci. USA 89:5547-5551; Oliva et al. (1992) Antimicrob. Agents Chemother. 36:913-919; Hlavka et al. (1985) Handbook of Experimental Pharmacology, Vol. 78 ( Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

In one embodiment, the overexpressed polynucleotide is targeted to the chloroplast for expression. In this manner, where the polynucleotide of interest is not directly inserted into the chloroplast, the expression cassette will additionally contain a nucleic acid encoding a transit peptide to direct the gene product of interest to the chloroplasts. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

In certain embodiments, the recombinant polynucleotide of the present invention can be stacked with any combination of polynucleotide sequences of interest in order to create plants with a desired trait. A trait, as used herein, refers to the phenotype derived from a particular sequence or groups of sequences. These stacked combinations can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

(F) Plants and Parts Thereof

Compositions of the invention further include plants, plant parts and plant cells having the recombinant polynucleotide of the invention. In specific embodiments, the recombinant polynucleotide is stably integrated into the genome of the plant, plant part or plant cell.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

II. Methods

The introduction and expression of the recombinant polynucleotide of the invention in a plant allows for an increase in the level of a first polynucleotide sequence of interest and further allows for a reduction or an elimination of the level of a second polynucleotide of interest or polypeptide encoded thereby. In specific embodiments, the silencing element employed reduces the level of an agronomically important sequence. In other embodiments, the overexpressed polynucleotide is also an agronomically important sequence.

A “modulated level” or “modulating the level” of a polynucleotide or a polypeptide in the context of the methods of the present invention refers to any increase or decrease in the expression, concentration, and/or activity of a gene product (i.e., polypeptide or polynucleotide), including any relative increment in expression, concentration and/or activity. The term “expression” as used herein in the context of a gene product, refers to the biosynthesis of that product including the transcription or translation of the gene product. In general, the level of the polypeptide or the polynucleotide is increased or decreased by at least 1%, 5%, 10%, 20%, 30%, 40%, 50%, 60%, 70%, 80%, 90%, 120%, or greater relative to a native control plant, plant part, or cell. Modulation in the present invention may occur during and/or subsequent to growth of the plant to the desired stage of development. In specific embodiments, the polynucleotides or the polypeptides of the present invention are modulated in monocots, particularly maize.

A “subject plant or plant cell” is one in which genetic alteration, such as transformation, has been effected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

The expression level of a polypeptide and/or an RNA may be measured directly, for example, by assaying for the level of the polypeptide or the RNA in the plant, or indirectly, for example, by measuring the activity of the polypeptide or the RNA in the plant, or by measuring a metabolite of the polypeptide or the end phenotype of the plant.

In specific embodiments, the recombinant polynucleotide of the invention is introduced into the plant cell. Subsequently, a plant cell having the introduced sequence of the invention is selected using methods known to those of skill in the art such as, but not limited to, Southern blot analysis, DNA sequencing, PCR analysis, or phenotypic analysis. A plant or plant part altered or modified by the foregoing embodiments is grown under plant forming conditions for a time sufficient to modulate the concentration and/or activity of polypeptides of the present invention in the plant. Plant forming conditions are well known in the art and discussed briefly elsewhere herein.

(A) Methods of Introducing the Recombinant Polynucleotide into a Plant or Part Thereof

Methods of introducing a polynucleotide into a plant are known and can be used to generate the plants, plant cells, and plant parts of the invention. “Introducing” is intended to mean presenting to the plant the polynucleotide in such a manner that the sequence gains access to the interior of a cell of the plant. The methods of the invention do not depend on a particular method for introducing a sequence into a plant, only that the polynucleotide gains access to the interior of at least one cell of the plant. Methods for introducing polynucleotides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polynucleotides into plants may vary depending on the type of plant or plant cell, i.e., monocot or dicot, targeted for transformation. Suitable methods of introducing polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the recombinant polynucleotide constructs of the invention can be provided to a plant using a variety of transient transformation methods. The polynucleotide construct can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which its released to become integrated into the genome is greatly reduced. Such methods include the use particles coated with polyethylimine (PEI; Sigma #P3143).

In other embodiments, the recombinant polynucleotide of the invention may be introduced into plants by contacting plants with a virus or viral nucleic acids. Generally, such methods involve incorporating a nucleotide construct of the invention within a viral DNA or RNA molecule. It is recognized that the recombinant polynucleotide of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a recombinant polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The recombinant polynucleotide of the invention is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a recombinant polynucleotide of the invention, for example, an expression cassette of the invention, stably incorporated into their genome.

(B) Modulating Tocol Content and Oil Production and/or Phytic Acid Level

In one embodiment of the invention, a recombinant polynucleotide of the invention is introduced into a plant. The recombinant polynucleotide comprises a first polynucleotide of interest, which when overexpressed, modulates tocol content. The first polynucleotide of interest further comprises an intron having an ADR-glucose Pyrophosphorylase (AGP) silencing element. While any promoter could be used to expresses the recombination polypeptide, in a specific embodiments, the recombinant polynucleotide is operably linked to an embryo-specific promoter. In specific embodiments, the first polynucleotide of interest comprises HGGT or a biologically active variant or fragment thereof, while the silencing element is an AGP silencing element. In more specific embodiments, the AGP silencing element is an AGP2 silencing element. Plants or plant parts expressing this recombinant polynucleotide are characterized as having an increase in tocol content, a disruption of starch biosynthesis and/or an increase in oil content. Such plants and plant parts find use in food and feed applications.

In one embodiment, the plants and plant parts produced by the methods of the invention have an increased efficiency of the wet or dry milling process.

In still another embodiment, a recombinant polynucleotide of the invention which is introduced into a plant comprises a first polynucleotide of interest, which when overexpressed, modulates tocol content. The first polynucleotide of interest further comprises an intron having a phytic acid silencing element. In specific embodiments, the first polynucleotide of interest comprises HGGT or a biologically active variant or fragment thereof, while the silencing element is any phytate silencing element, such as a LPA1 silencing element, a LPA2 silencing element, and/or a LPA3 silencing element. Plants or plant parts expressing this recombinant polynucleotide are characterized as having an increase in tocol content and a reduced phytic acid level. Such plants find use in food and feed applications.

In still another embodiment, a recombinant polynucleotide of the invention which is introduced into a plant comprises a first polynucleotide of interest, which when overexpressed, modulates tocol content. The first polynucleotide of interest further comprises an intron having a FAD silencing element. In specific embodiments, the first polynucleotide of interest comprises HGGT, while the silencing element is any FAD silencing element, such as a FAD2 silencing element. Plants or plant parts expressing this recombination polynucleotide are characterized as having an increase in tocol content and an elevated level of oleic acid. Such plants find use in food and feed applications

It is further recognized that more than one silencing element can be employed in the methods of the invention. Accordingly, any one of the FAD silencing elements and/or AGP silencing elements and/or phytic acid silencing elements can be used alone or in combination with another silencing element in the recombinant polynucleotide to modulate the desired agronomically important traits.

(D) Modulating Amino Acid Compositions of a Plant or Plant Part

In one embodiment of the invention, a recombinant polynucleotide of the invention is introduced into a plant. The recombinant polynucleotide comprises a first polynucleotide of interest, which when overexpressed, increases the amino acid composition of the plant or plant part. The first polynucleotide of interest further comprises an intron having a prolamin silencing element. In specific embodiments, the first polynucleotide of interest comprises any BHL polypeptide or functional variant or fragment thereof, while the silencing element is a prolamin silencing element (i.e., a α-zein, β-zein, γ-zein, and/or δ-zein silencing element). In specific embodiments, the BHL polypeptide is BHL9 or a biologically active variant thereof and the prolamin silencing element comprises a 27 kD γ-zein silencing element and/or a 50 kD γ-zein silencing element and/or an LKR silencing element. Plants or plant parts expressing this recombinant polynucleotide are characterized as having improved amino acid composition/nutrient value of the seed, improved digestibility and nutrient availability, improved response to feed processing and improved silage quality.

It is further recognized that more than one silencing element can be employed in the methods of the invention. Accordingly, any one of the prolamin silencing elements, can be used alone or in combination with another prolamin silencing element in the recombinant polynucleotide.

While any promoter could be used to expresses the recombination polypeptide, in specific embodiments, the recombinant polynucleotide is operably linked to an endoderm-specific promoter.

The following examples are offered by way of illustration and not by way of limitation.

### EXPERIMENTAL

### Example 1

### Modulating the Level of Red Fluorescent Protein and Phytoene Desaturase

A DNA construct comprising a spliceable intron (ST-LS1 INTRON2) was cloned into the coding sequence of DS-RED2 (red fluorescent protein, RFP) such that only accurate splicing of the intron would restore the correct open reading frame and allow translation of functional RFP. Within this intron, an inverted repeat sequence from the coding sequence of maize phytoene desaturase (ZM-PDS1) which had been demonstrated to silence expression of endogenous PDS when expressed alone as a hair-pin containing transcript. The chimeric transcript was placed under the transcriptional control of a maize ubiquitin promoter::ubiqutin intron. The construct was introduced into immature maize embryos using a linked herbicide resistance marker for selecting transformation events. Somatic embryos regenerated into plantlets under evaluated light conditions showed photobleaching characteristics of PDS silencing. Photobleached plantlets were also positive for RFP, suggesting the accurate splicing of the intron.

### Example 2

### Modulating Tocol Content and Oleic Acid and Phytic Acid Content in Maize

A DNA construct is generated comprising the HV-HGGT coding sequence for the overexpression of the HGGT polypeptide which is interrupted by a spliceable intron which itself includes a silencing element for FAD2 and/or any or all of the phytate genes (LPA1, LPA2, LPA3). The DNA construct is operably linked to an embryo-specific promoter.

Immature maize embryos from greenhouse donor plants are bombarded with a plasmid containing the DNA construct described above and the selectable marker gene PAT (Wohlleben et al. (1988) Gene 70:25-37), which confers resistance to the herbicide Bialaphos. Alternatively, the selectable marker gene is provided on a separate plasmid. Transformation is performed as follows. Media recipes follow below.

Preparation of Target Tissue

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

A plasmid vector comprising the DNA construct described above is made. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂; and, 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #4 in particle gun #HE34-1 or #HE34-2. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for an elevated level of tocotrienols (from the expression of HV-HGGT) plus an elevated level of oleic acid (from the suppression of FAD2) and/or reduced phytic acid levels (suppression of phytate genes).

Bombardment medium (560Y) comprises 4.0 g/l N6 basal salts (SIGMA C-1416), 1.0 ml/l Eriksson's Vitamin Mix (1000X SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000X SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos(both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 3

### Modulating the Amino Acid Composition of a Plant or Plant Part

A DNA construct is generated comprising the synthetic balanced amino acid storage protein BHL9 coding sequence for the overexpression of the BHL9 polypeptide which is interrupted by a spliceable intron which itself includes silencing sequences (sense, antisense, hairpin sequences) for any or all of the alpha zeins, 27 kd gamma zein, 50 kd gamma zein, and LKR. The DNA construct is operably linked to an endosperm-specific promoter.

For Agrobacterium-mediated transformation of maize with the recombinant polynucleotide described above, the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the recombinant polynucleotide to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants.

The plants are scored for an improved amino acid composition.

Soybean embryos are bombarded with a plasmid containing a DNA construct is generated comprising the synthetic balanced amino acid storage protein BHL9 coding sequence for the overexpression of the BHL9 polypeptide which is interrupted by a spliceable intron which itself includes silencing sequences (sense, antisense, hairpin sequences) for any or all of the alpha zeins, 27 kd gamma zein, 50 kd gamma zein, and LKR. The DNA construct is operably linked to an endosperm-specific promoter.

To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the GM-ALS (Acetolactase synthase allele) operably linked to the GM-SAMs promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the-same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A recombinant polynucleotide comprising a first agronomically important polynucleotide comprising an intron comprising a silencing element, wherein
said intron is capable of being spliced in a plant from said first agronomically important polynucleotide;
upon splicing of said intron, the expression of the first agronomically important polynucleotide is increased; and,
said intron is capable of reducing the level of a second agronomically important polynucleotide or a polypeptide encoded thereby.

2. The recombinant polynucleotide of claim 1, wherein said silencing element comprises a hairpin suppression element.

3. The recombinant polynucleotide of claim 1, wherein said recombinant polynucleotide is operably linked to a promoter active in the plant.

4. The recombinant polynucleotide of claim 3, wherein said promoter comprises a constitutive promoter, a tissue-specific promoter, a developmentally-regulated promoter, or an inducible promoter.

5. The recombinant polynucleotide of claim 4, wherein said promoter comprises an endosperm-specific promoter or an embryo-specific promoter.

6. The recombinant polynucleotide of claim 1, wherein said first or said second agronomically important polynucleotide modulates tocol content, oleic acid content, phytic acid content, amino acid composition, oil quality or quantity, energy availability, digestibility, fatty acid composition, a pathogen defense mechanism, lysine and sulfur levels, starch synthesis, disease resistance, herbicide resistance, male sterility, plant vigor, nutrient content, yield, growth pattern, digestibility and energy value of the grain, hemicellulose content, cellulose production, or tolerance to salt, heat, drought and/or cold tolerance.

7. The recombinant polynucleotide of claim 6, wherein said first agronomically important polynucleotide comprises homogentisate geranylgeranyl transferase (HGGT) and said second agronomically important polynucleotide comprises ADR-glucose Pyrophosphorylase 2 (AGP2), Fatty Acid Desaturase 2 (FAD2), Low Phytic Acid 1 (LPA1), Low Phytic Acid 2 (LPA2), or Low Phytic Acid 3 (LPA3).

8. The recombinant polynucleotide of claim 7, wherein said recombinant polynucleotide is operably linked to an embryo-specific promoter.

9. The recombinant polynucleotide of claim 6, wherein the first agronomically important polynucleotide comprises a storage protein.

10. The recombinant polynucleotide of claim 9, wherein the first agronomically important polynucleotide comprises Barley High Lysine 9 (BHL9) and said second agronomically important polynucleotide comprises a 27 kD gamma zein polynucleotide, a 50 kD gamma zein polynucleotide, an alpha zein polynucleotide, or a LKR polynucleotide.

11. The recombinant polynucleotide of claim 10, wherein the recombinant polynucleotide is operably linked to an endosperm-specific promoter.

12. A vector comprising the recombinant polynucleotide of claim 1.

13. A plant or plant part comprising the recombinant polynucleotide of any one of claim 1.

14. The plant or plant part of claim 13, wherein said recombinant polynucleotide is stably integrated into the genome of the plant.

15. A seed having stably incorporated in its genome the recombinant polynucleotide of claim 1.

16. A method for modulating the expression of at least two polynucleotides of interest in a plant comprising
a) introducing into the plant a recombinant polynucleotide comprising a first polynucleotide of interest comprising an intron comprising a silencing element, wherein
i) said intron is capable of being spliced from said first polynucleotide of interest in the plant;
ii) upon splicing of said intron, the expression of the first polynucleotide of interest is increased; and,
iii) said intron is capable of reducing the expression of a second polynucleotide of interest or a polypeptide encoded thereby; and,
b) expressing said recombinant polynucleotide in said plant or a plant part and thereby increasing the expression of the first polynucleotide of interest and reducing the expression of the second polynucleotide of interest or a polypeptide encoded thereby.

17. The method of claim 16, wherein said second polynucleotide of interest confers an agronomically important trait.

18. The method of claim 17, wherein said first polynucleotide of interest confers an agronomically important trait.

19. The method of claim 16, wherein said silencing element comprises a hairpin suppression element.

20. The method of claim 16, wherein said recombinant polynucleotide is operably linked to a promoter active in the plant.

21. The method of claim 20, wherein said promoter comprises a constitutive promoter, a tissue-specific promoter, a developmentally-regulated promoter, or an inducible promoter.

22. The method of claim 21, wherein said promoter comprises an endosperm-specific promoter or an embryo-specific promoter.

23. The method of claim 16, wherein said first or said second polynucleotide of interest modulates tocol content, oleic acid content, phytic acid content, amino acid composition, oil quality or quantity, energy availability, digestibility, fatty acid composition, a pathogen defense mechanism, lysine and sulfur levels, starch synthesis, disease resistance, herbicide resistance, male sterility, plant vigor, nutrient content, yield, growth pattern, digestibility and energy value of the grain, hemicellulose content, cellulose production, or tolerance to salt, heat, drought and/or cold tolerance.

24. The method of claim 23, wherein said first agronomically important polynucleotide comprises HGGT homogentisate geranylgeranyl transferase (HGGT) and said second agronomically important polynucleotide comprises ADR-glucose Pyrophosphorylase 2 (AGP2), Fatty Acid Desaturase 2 (FAD2), Low Phytic Acid 1 (LPA1), Low Phytic Acid 2 (LPA2), or Low Phytic Acid 3 (LPA3).

25. The method of claim 24, wherein said recombinant polynucleotide of interest is operably linked to an embryo-specific promoter.

26. The method of claim 23, wherein the first polynucleotide of interest comprises a storage protein.

27. The method of claim 26, wherein the first polynucleotide of interest comprises Barley High Lysine 9 (BHL9) and said second agronomically important polynucleotide comprises a 27 kD gamma zein, a 50 kD gamma zein, an alpha zein, or LKR.

28. The method of claim 27, wherein the recombinant polynucleotide is operably linked to an endosperm-specific promoter.

29. A plant or plant part comprising a recombinant polynucleotide comprising a first polynucleotide of interest comprising an intron comprising a silencing element, wherein
said intron is capable of being spliced in the plant from said first polynucleotide of interest;
upon splicing of said intron, the expression of the first polynucleotide of interest is increased; and,
said spliced intron is capable of reducing the level of a second polynucleotide of interest or a polypeptide encoded thereby.

30. The plant or plant part of claim 29, wherein said recombinant polynucleotide is stably integrated into the genome of the plant.

31. A seed of the plant of claim 30, wherein said seed has stably incorporated into its genome the recombinant polynucleotide.
